(19) United States
(12) Patent Application Publication
(10) Pub. No.: US 2022/0099674 A1
(43) Pub. Date: Mar. 31, 2022

(54) RAPID TEST SYSTEM FOR VIRAL AND BACTERIAL INFECTIONS

(71) Applicant: The Cleveland Clinic Foundation, Cleveland, OH (US)

(72) Inventors: David Geoffrey Vince, Westlake, OH (US); Aaron J. Fleischman, Beachwood, OH (US); Feng Lin, Orange, OH (US)

(21) Appl. No.: 17/488,384

(22) Filed: Sep. 29, 2021

Related U.S. Application Data
(60) Provisional application No. 63/085,404, filed on Sep. 30, 2020.

(51) Int. Cl. G01N 33/569; G01N 1/22; G01N 33/543; G01N 33/58; B01L 3/00

(52) U.S. Cl. CPC G01N 33/56983; B01L 2400/0478; G01N 33/54333; G01N 33/582; B01L 3/502761; G01N 2001/2244; G01N 2333/165; B01L 2200/027; B01L 2200/0668; B01L 2200/0684; B01L 2200/082; B01L 2200/10; B01L 2200/16; B01L 2300/023; B01L 2300/027; B01L 2300/0609; B01L 2300/0663; B01L 2300/0681; B01L 2300/161; G01N 1/2214

(57) ABSTRACT

A method for detecting viral or bacteriological pathogens is disclosed. The method comprises collecting a potentially pathogenic sample via a collector, binding a first portion of the potentially pathogenic sample to a magnetic particle via a first coating on the magnetic particle, binding a second portion of the potentially pathogenic sample to a fluorescently labeled particle via a coating of a second coating on the fluorescently labeled particle to create aggregates comprising the potentially pathogenic sample, magnetic particle, and the fluorescently labeled particle, separating the aggregates magnetically, detecting a fluorescence of the separated aggregates, and estimating an amount of the pathogen based on the detected fluorescence.

### RELATED APPLICATIONS

This application claims priority to U.S. Provisional Patent Application No. 63/085,404, filed on Sep. 30, 2020 and titled “Rapid Test System for Viral and Bacterial Infections,” the entire contents of which are herein incorporated by reference.

### FIELD

This disclosure relates generally to testing of virus and bacterial infections, particularly rapid testing using breathalyzer systems. It also relates to systems and methods for pathogen identification and quantification, particularly those using fluorescent and magnetic labeling and micro electromechanical systems (MEMS).

### BACKGROUND

Recent global pandemics, including COVID-19, have shown the need for rapid testing and screening of individuals for infection. Preventing or slowing the spread of disease requires reliably identifying and isolating infected individuals before they can infect others. This can be especially difficult when the infected can be asymptomatic, as in the case of COVID-19.

Currently, there is a lack of rapid, accurate and low-cost diagnostic testing for the SARS-CoV-2 virus and other pathogens. Laboratories have deployed quantitative reverse transcription-polymerase chain reaction (qRT-PCR) assays for virus detection. However, qRT-PCR turnaround times for screening and diagnosing patients can range from hours to days. These tests are also expensive and require skilled technicians to complete. Shortages of relevant skills can also lead to bottlenecks in processing.

Most of the tests currently under development by major healthcare companies focus on rapid diagnostic screening in a laboratory environment and are therefore unsuitable for point-of-care (POC) applications. These approaches include molecular testing using real-time PCR, Droplet Digital PCR, and immunological assays. Many tests claimed to be POC must still be performed at a healthcare facility, as samples are taken by swabbing the nasopharyngeal, nasal, mid-turbinate, or oropharyngeal regions of the patient. This is both challenging for the healthcare worker and uncomfortable for the patient. As of writing, only one such test has received emergency use authorization (EUA) from the FDA for home sample collection. The test kit, which includes a nasal swab and saline for self-collection, must be returned to a clinical laboratory for analysis and is therefore not rapid. Swab samples are difficult to obtain and can reduce the sensitivity of the test, leading to false negative results. There is an urgent need for a POC test, particularly for SARS-CoV-2, that can be completed while a patient is on-site at the testing location, that can be administered and processed without the need for specialized equipment, and is cost-effective enough for use in economically disadvantaged areas or countries.

Faster tools for identifying and quantifying pathogens do exist, but are mainly limited to laboratory environments. For example, fluorescent molecules or particles can be joined to antibodies that can bind to the pathogens. This labels pathogens with fluorescent markers so they can be detected and quantified. Similarly, magnetic particles can be joined to cells via antibodies. The particles can then be separated and analyzed via magnetic field and/or fluorescence imaging.

As discussed in more detail in U.S. Pat. No. 6,623,894 to Fleischman et al., herein incorporated by reference in its entirety, labeled particles can be manipulated and quantified using MEMS technology that creates high-precision microscopic structures on silicon and other materials. Through MEMS technology, relatively low-cost, high-volume, electromechanical systems that integrate sensors, actuators, electronics, and other components can be realized on a miniature scale. Related technologies have been able to detect malaria-infected red blood cells using a MEMS microfluidic magnetic separator test. See P. A. Zimmerman, J. M. Thomson, H. Fujioka, W. E. Collins, and M. Zborowski, “Diagnosis of malaria by magnetic deposition microscopy,” Am. J. Trop. Med Hyg., vol. 74, pp. 568-72, April 2006. The technology successfully separated cells and particles that exhibit far weaker magnetic properties than those labeled with magnetic markers, as described above. U.S. Pat. No. 5,968,820 to Zborowski et al., hereby incorporated by reference in its entirety, describes methods and apparatuses for magnetically separating cells into fractionated flow streams. Through a combination of flow compartments and one or more magnetic fields, a flow stream of heterogeneous cells is separated into fractionated flow streams based on cell magnetic dipole moment and analyzed. However, to date, these and related technologies have not yet been integrated in a convenient, cost-effective, and rapid screening test for the pathogens themselves.

For at least the above reasons, there is currently a need for a rapid viral or pathogen screening test. Advantageously, such a test would sample the patient's breath, which can be collected relatively quickly and with minimal invasiveness. Ideally, the test would be sensitive to relatively small amounts of pathogen and provide relatively fast results without the use of extensive laboratory analysis. It would incorporate diagnostics into a single, disposable package using MEMS technology.

### SUMMARY

Disclosed herein is a method for detecting viral or bacteriological pathogens. The method comprises collecting a potentially pathogenic sample via a collector, binding a first portion of the potentially pathogenic sample to a magnetic particle via a first coating on the magnetic particle, binding a second portion of the potentially pathogenic sample to a fluorescently labeled particle via a coating of a second coating on the fluorescently labeled particle to create aggregates comprising the potentially pathogenic sample, magnetic particle, and the fluorescently labeled particle, separating the aggregates magnetically, detecting a fluorescence of the separated aggregates, and estimating an amount of the pathogen based on the detected fluorescence.

The separating may be according to at least one of distances traveled by the aggregates in the microfluidic magnetic separator, times of travel of the aggregates in the microfluidic magnetic separator, and flow of the aggregates in the microfluidic magnetic separator.

The estimating an amount of the pathogen based on the detected fluorescence may comprise estimating the amount based on a spatial distribution of the detected fluorescence in the microfluidic magnetic separator.

The spatial distribution of the detected fluorescence may result from the separating.

The detecting a fluorescence of the separated aggregates may comprise illuminating the aggregates, an detecting an amount of fluorescence of the fluorescently labeled particles excited by the illumination.

The florescence may be a fluorescence of the fluorescently labeled particles in the aggregates. The method may comprise estimating a viral load in the patient based on the estimated amount of pathogen.

Also disclosed herein is a device for detecting viral or bacteriological respiratory pathogens. The device comprises a breath capture portion for capturing a potentially pathogenic sample. The breath capture portion comprises a mouthpiece, an inlet tube, and a collection tank connected to the mouthpiece via the inlet tube. The collection tank comprises magnetic particles coated with a first coating that binds to a first portion of a pathogen and fluorescently labeled particles coated with a second coating that binds to a second portion of the pathogen. The device further comprises an outlet tube connecting the collection tank to a microfluidic channel, the microfluidic channel forming part of a microfluidic magnetic separator.

At least one of the breath capture portion may comprise a window configured to pass fluorescent light from the microfluidic channel to outside the breath capture portion, and the window may be configured to allow light from outside the breath capture portion to illuminate the microfluidic channel.

The device may comprise a detection system configured to detect, through the window, fluorescence over a range of fluorescently labeled particle displacements in the microfluidic channel, and provide, through the window, light to the microfluidic channel. The detection system may comprise an LED detection system that illuminates the fluorescently labeled particles with LED light. The coronavirus may be SARS-CoV-2, and the first portion of SARS-CoV-2 to which the first coating binds and the second portion of SARS-CoV-2 to which the second coating binds may be different epitopes of a SARS-CoV-2 spike protein. The second coating may comprise angiotensin converting enzyme 2 (ACE2).

At least one surface of the inlet tube and outlet tube may be hydrophobic, the device may comprise a filter positioned to remove debris from the potentially pathogenic sample, the device may comprise an exhaust filter that removes pathogen from vapor to be expelled from the device. A part of the breath capture portion is disposable. At least one of the disposable portion may be the mouthpiece, the mouthpiece is detachable from the breath capture portion, and the entire breath capture portion may be disposable.

The device may comprise a base, separate from the breath capture portion, comprising an interface for physically accommodating at least a portion of the breath capture portion, electronics configured to obtain fluorescence data from the breath capture portion, and a communications port for transferring communications based on the fluorescence data.

At least one of the electronics may comprise a detection system for detecting the fluorescence data, the electronics comprise at least one of a video camera and a photosensor, the at least one of a video camera and a photosensor is positioned to detect fluorescence over a range of fluorescently labeled particle displacements within the microfluidic channel, the electronics is configured to run software to analyze images of the fluorescence, the communications port comprises at least one of an ethernet port, a Bluetooth connection, a WiFi connection, a mobile phone connection, a bar-code reader or other method to correlate patient and breath sample, and an optical display on the base, the base further comprises a piston positioned to be in mechanical communication with a part of the breath capture portion, the part of the breath capture portion in mechanical communication with the piston comprises a plunger configured to displace the potentially pathogenic sample within the breath capture portion, the piston is configured to actuate the plunger to displace the potentially pathogenic sample within the breath capture portion, the base further comprises a motor configured to actuate the piston, and the microfluidic magnetic separator has a High Gradient Magnetic Separation (HGMS) configuration.

The microfluidic magnetic separator may have an Open Gradient Magnetic Separation (OGMS) configuration.

Advantages of the disclosed device and method include that they provide the only breath-based SARS-CoV-2 test to date, and the only such test that combines the advantages of immunoassay and microfluidics. Its breathalyzing based test is more economical, faster, more comfortable for the consumer, and easier to perform than currently developed tests. Collected samples are of more uniform quality. Analysis requires little to no laboratory testing and can be completed at POC virtually automatically. In addition, portions of the device (e.g., the breathalyzer portion) are disposable, improving hygiene, efficiency, and rapidity. Portions of the device may be fabricated simply and cheaply using techniques such as lamination, injection molding, and/or 3D printing. Also, the power requirements of the device are low enough such that it can be battery driven.

### BRIEF DESCRIPTION OF DRAWINGS

FIG. 1 shows a pathogen collection and analysis system 100 according to aspects of the present disclosure.

FIG. 2A shows sample collection portion 150 of system 100 removed from the interface or accommodator 112 of the analyzer or base 110.

FIG. 2B shows an exemplary channel 168 and magnet 170 setup 600 located inside portion 150.

FIG. 3 presents an exemplary pathogen labeling system 300 that may be used in conjunction with system 100.

FIG. 4 shows another pathogen labeling system 400 that may be used in conjunction with system 100.

FIG. 5A shows systems 300 and 400 combined in system 500 for labeling testing targeted pathogens 510.

FIG. 5B shows a schematic of an aggregate 520 where the magnetic particle 310 and the fluorescently labeled particle 410 are bound to different subunits S1 and S2, respectively, of a spike protein on pathogen 510.

FIG. 6A shows a schematic of a microfluidic channel 168 and magnet 170 portion of system 100 in one exemplary variation.

FIG. 6B shows a photograph of an actual implementation of the microfluidic channel 168 and magnet 170 of FIG. 6B as microfluidic magnetic separator 650.

FIG. 7A shows a schematic of the operation of an exemplary microfluidic magnetic separator configuration 700 in conjunction with system 100.

FIGS. 7B-7D show operation of another exemplary microfluidic magnetic separator configuration 750 in conjunction with system 100.

FIG. 8 shows a schematic of the operation of another exemplary microfluidic magnetic separator configuration 800 in conjunction with system 100.

FIGS. 9A and 9B are a flowchart of a method for using system 100 to detect pathogens in a patient.

### DETAILED DESCRIPTION

Several illustrative embodiments will be described in detail with the understanding that the present disclosure merely exemplifies the general inventive concepts. Embodiments encompassing the general inventive concepts may take various forms and the general inventive concepts are not intended to be limited to the specific embodiments described herein.

### Overview of the Testing System

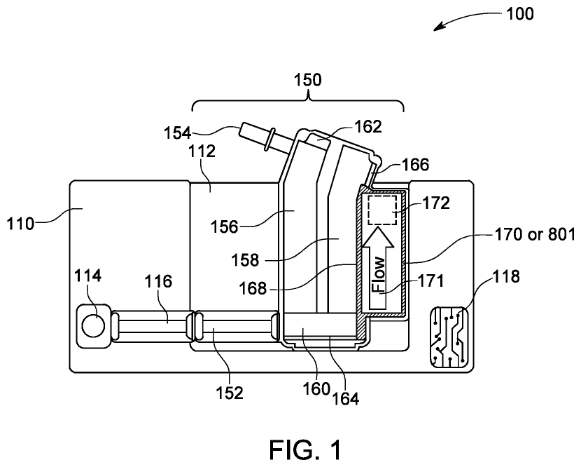

FIG. 1 shows a pathogen collection and analysis system 100 according to aspects of the present disclosure. The system 100 can be divided into two main portions, an analyzer base 110 and a sample collection portion 150. The sample collection portion 150 may be configured for obtaining a sample from a patient (e.g., via the patient's breath or saliva). The analyzer base 110 may then analyze the collected sample for pathogens and report a result. As shown in FIG. 1, the collection portion 150 and the analyzer base 110 may be separate components. In this case, one or more of the portions (e.g., the collection portion 150) may be designed for single use and to be disposable. The other portion (e.g., the analyzer base 110) may be designed for multiple uses with different collection portions 150. However, it is to be understood that such a configuration is merely exemplary. Collection portion 150 and analyzer base 110 may also be contained within the same housing. Both may be disposable.

As shown in FIG. 1, the analyzer base 110 may include an interface or accommodator 112 for accommodating the sample collection portion 150. The interface or accommodator 112 may include electrical and/or mechanical connections (not shown) to interface with the sample collection portion 150. The electrical connections, for example, may power portions of the sample collection portion 150 and/or retrieve data from collection portion 150. It may charge a battery (not shown) in the collection portion 150. The interface or accommodator 112 may also include a motor or actuator 114 that may drive a piston 116 that is also part of the interface or accommodator 112. The piston 116 may interface with a plunger 152 on the sample collection portion 150 for driving a sample with the sample collection portion 150, as discussed in more detail below.

Analyzer base 110 may further include electronics (e.g., power regulation electronics, communications electronics, application specific integrated circuits (ASICs) for the exemplary purposes of communications and/or data processing) symbolically represented as 118. Electronics 118 may drive such functions as piston 116 and motor 114 activation, strength of the magnetic field created by magnet 170 or magnet 801, and function of any measurement devices. It may run heating or cooling devices in portion 150 or base 110 and/or charge a battery on the collection portion 150. Electronics 118 may include central processing units (CPUs) and other hardware capable of performing the data analysis described herein. In addition or alternatively, base 110 may be connected such that it shares data directly with a computer or other device. Analyzer base 110 may be Bluetooth capable and/or be able to communicate results via ethernet, WiFi and/or cellphone communications systems. Electronics 118 may also include bar code reader capabilities for the collection and correlation of patient data.

As also shown in FIG. 1, sample collection portion 150 may include a sample extractor 154 for extracting patient samples for analysis (e.g., breathalyzing). For example, a patient may place his or her lips on the end of the sample extractor 154 and breath into inlet tube 156. The sample extractor 154 may be any suitable kind of receptacle, such as a tube, hole, nozzle, or mouthpiece. Breath intake portion 150 may also or alternatively be configured to obtain samples from the patient in other forms. For example, breath intake portion 150 may be configured to obtain saliva or mucus-based samples from the patient. Breath intake portion 150 may also be configured to obtain samples from other bodily fluids and/or materials. If portions of the sample collection portion 150 are not disposable, the sample extractor 154 may be detachable and disposable by itself.

Portion 150 may further include a series of tubes, such as inlet tube 156 and outlet tube 158, disposed on either side of collection tank 160. The tubes 156 and 158 may include filters for filtering debris from the sample. Tubes 156 and 158 may also include valves and/or baffles to retard or stop flow. Both tubes 156 and 158 may be hydrophobic (e.g., their interior walls may be coated with a hydrophobic coating and/or the tubes themselves may be entirely made of hydrophobic material) to facilitate flow of water-based breath, or other, samples throughout portion 150. Tubes 156 and 158 may have increased surface area for additional efficiency in collected exhaled samples. Portion 150 may further include an emission device (e.g., pouch) 162 that may eject saline, other liquid, and/or gas to propel a breath sample from the sample extractor 154 through the inlet tube 156. Any suitable type of propellant may be used in conjunction with emission device 162. Suitable types of propellant are generally those that will not substantially interfere, confound, or degrade the accuracy of the various measurement techniques (e.g., the magnetic and/or fluorescent measurement techniques discussed in more detail below).

Collection tank 160 generally holds reagents 164, potentially including magnetically and/or fluorescently tagged antibodies and/or particles, as discussed in more detail blow. Reagents 164 may have a number of purposes, one being to facilitate the collection and analysis of pathogen samples from the patient sample provided by the sample extractor 154. Collection tank 160 may also include chemicals that stabilize and/or preserve the various regents before use. In variations in which the collection portion 150 can be used multiple times, the chemicals in collection tank 160 may stabilize and/or preserve the reagents between uses. Regents may be stored in powder or other solid form (e.g., as lyophilized powder) and injected or released to the sample or other carrying liquid upon use of the collection portion 150. Reagents may also be stored in liquid (e.g., aqueous) form. Certain reagents may be stored and/or supplied as gases.

Collection tank 160 may include or be adjacent to a cooler, such as a Peltier cooler. Other coolers may also be used, including various electric coolers, dry ice, liquid nitrogen, “blue ice gel” (typically pre-cooled or pre-frozen) and/or a disposable cooler cartridge with one or more of these elements. The cooler may be located in the base 110 and be in thermal communication with tank 160, or it may be located on portion 150. The cooler may preserve the reagents as well as allow condensing or consolidation of the breath sample. One of the functions of the cooler is to condense breath samples into a breath condensate. Among other things, this can help the collector portion 150 obtain amounts of breath sample sufficiently for accurate testing.

After a patient blows into or otherwise provides a sample to sample extractor 154, the sample enters inlet tube 156. The sample may settle to the collection tank 160 under the force of gravity and/or pressure. The sample may also or alternatively be propelled by emission device 162 into collection tank 160, as described in more detail below. Once the sample is in the collection tank 160, it may bond to the reagents 164 for later identification. It also may be condensed by the aforementioned cooler to form exhaled breath condensate (EBC) for improved measurement sensitivity and detection.

Parts of the sample that do not collect in collection tank 160 may be transitioned through outlet tube 158. The portions of the breath sample that flow this way are the uncaptured sample portions. The uncaptured sample portions may be filtered by exhaust filter 166. Exhaust filter 166 may remove pathogens (e.g., viruses) in the uncaptured sample portions before releasing them to the ambient environment outside the collection portion 150. Exhaust filter 166 may be constructed of any suitable filter materials, including but not limited to HEPA type filters that can block or avoid spreading aerosolized respiratory pathogens.

Piston 116 in the base 110 may actuate the collection portion's 150 plunger 152 to push the captured portions of the breath sample through the collection tank 160 and into the microfluidic channel 168. Pistons 116 and 152 may be in mechanical communication, while only plunger 152 is in fluid communication with the contents inside tube 156, potentially including a breath sample taken from a patient. Action of piston 116 and plunger 152 may propel a condensed (or other) breath sample from collection tank 160 up through microfluidic channel 168 into the vicinity of an analytic system (e.g., the various microfluidic magnetic separators discussed herein). Specifically, plunger 152 can actuate flow of sample upward and against gravity along the “Flow” labeled path in microfluidic channel 168. In this way the sample can flow from the bottom of system 100 upward.

Plunger 152 may also be used to “prime” the portion of system 100 (e.g., the analytical portion) in communication with microfluidic channel 168. For example, plunger 152 may push fluid into channel 168 before the sample is added and/or before analysis is performed in order to displace gas (e.g., air) and/or gas bubbles that may have formed in the channel 168 or elsewhere in device 100. The gas can be expelled out of exhaust 166, for example, or some other exit mechanism. It is often advantageous to eliminate bubbles in channel 168. Bubbles can prevent or hinder fluid flow. They may also confound certain types of measurements. Therefore, it is often useful to prime the system to remove or expel bubbles. It is to be appreciated that other mechanisms than plunger 152 may be used to prime the system and remove bubbles in this way. Any suitable configuration for priming is within the scope the present disclosure (e.g., a pump system or other system that can actuate fluid flow).

Although FIG. 1 shows flow in one direction (“Flow”) due to action of plunger 152, in fact this direction of flow can be reversed. For example, sample may flow downward in the opposite direction of the labeled “Flow” due to gravity. Also, plunger 152 may be withdrawn, causing liquid to withdraw and create a fluid flow in the opposite direction of “Flow.” This reverse flow may be initiated deliberately. In some instances, it may be advantageous to extend and retract plunger 152 cyclically in order to create a cyclic flow of sample in channel 168. Doing so may help collect and accumulate sample in the analytic portions of the system 100 discussed further below. It may also allow good mixing of the sample with any carrier fluid or other components of the system described herein. The cycling may further improve the amount of sample exposed to the analytic portion of system 100 and, therefore, increase or augment the signal to noise ratio of the measurement. This cycling process may be assisted by a sample collection portion or reservoir (not shown) that can be located toward the top of system 100 (e.g., close to filter 166) for collecting sample near the top of system 100. This collection portion or reservoir can provide sample for the flow cycling just mentioned. In addition, the collection portion or reservoir can simply hold sample for later removal from system 100 for further analysis (e.g., for situations in which more than one analytical technique is advantageous and/or for repeat analysis) and/or for storage.

As shown in FIG. 1, microfluidic channel 168 may be aligned with magnet 170 or 801, for magnetic separation and detection as discussed in more detail below. Flow arrow 171 gives an exemplary direction of flow of sample within the microfluidic channel 168, as shown in FIG. 1. One of the methods of detection includes using detection system 172, which may be located on the base 110. In alternative configurations, detection system 172 may be located on the portion 150. System 172 may detect particles via fluorescence of fluorescently labeled antibodies, molecules, and/or synthetic or other particles. FIG. 1 shows an exemplary position of the detection system 172. If the detection system 172 is located on the base 110, the adjacent portion of the collection portion 150 may have a window (not shown) so that light may be communicated from to and from the detection system 172 and the channel 168.

If the collection portion 150 may be used multiple times, then aspects of it may be switched out and replaced. As discussed above, the sample extractor 154 may be switched out and replaced for multiple uses. In addition, the collection tank 160 and reagents 164 may be replaced, as well as the exhaust filter 166. Portions of these components (e.g., the collection tank 160 and reagents 164) may be replaced in modular or cartridge form. During the replacement, exposed portions of the collection portion 150 may be covered with a disposable cover so that the interior of the portion 150 is not contaminated.

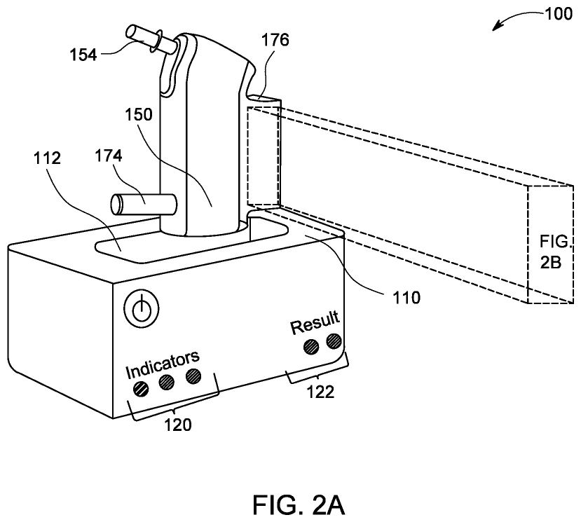

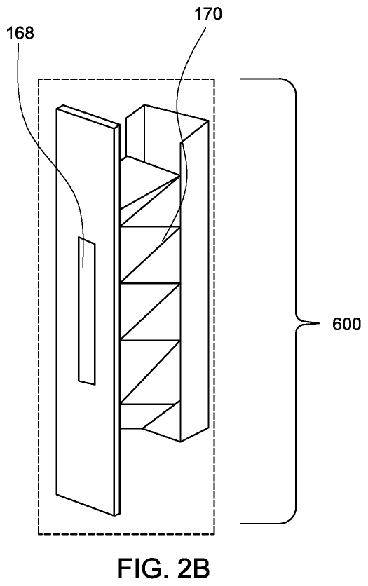

FIG. 2A shows sample collection portion 150 removed from the interface or accommodator 112 of the analyzer or base 110. In particular, FIG. 2A shows the exterior housing of plunger 152 of the sample collection portion 150. External housing 174 may form a fluid-tight seal over plunger 152, shielding plunger 152 from external elements. Housing 174 would thereby prevent contamination of samples and reagents 164 in the interior of portion 150 (e.g., samples within inlet tube 156 and/or collection tank 160). External housing 174 may be moved with plunger 152 in order to actuate fluid flow within portion 150, as discussed above. When external housing 174 moves with the plunger 152, it may maintain shielding of the interior of portion 150 from the elements and to shield piston 116 from the contents of portion 150. The latter prevents piston 116, which may not be disposable, from becoming contaminated with a breath sample inside collection portion 150. FIG. 2A further shows the exterior housing 176 of the magnet(s) 170 and microfluidic channel 168 of portion 150.

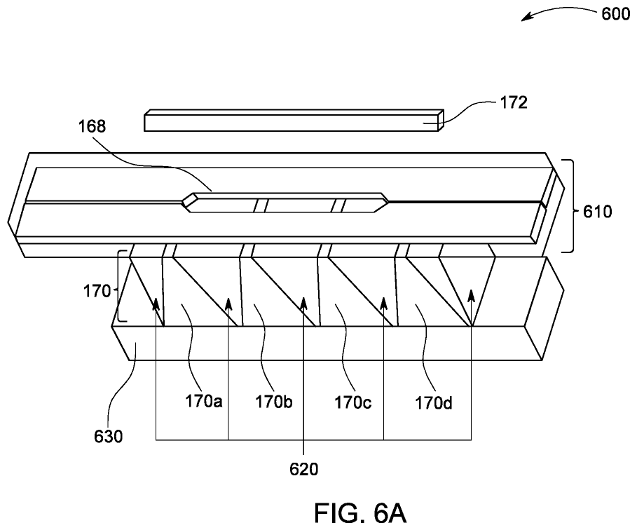

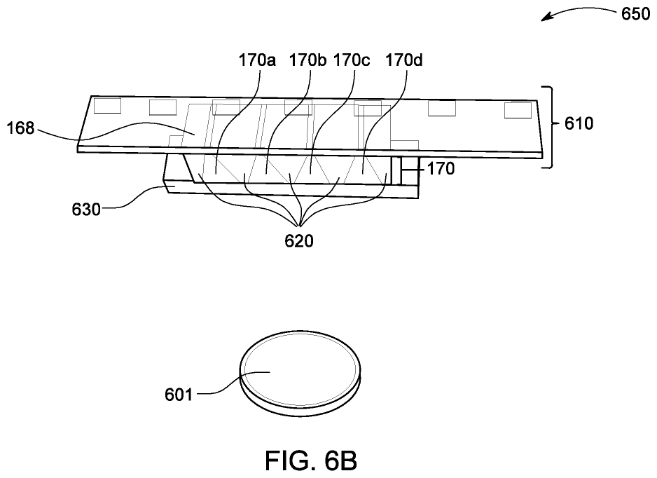

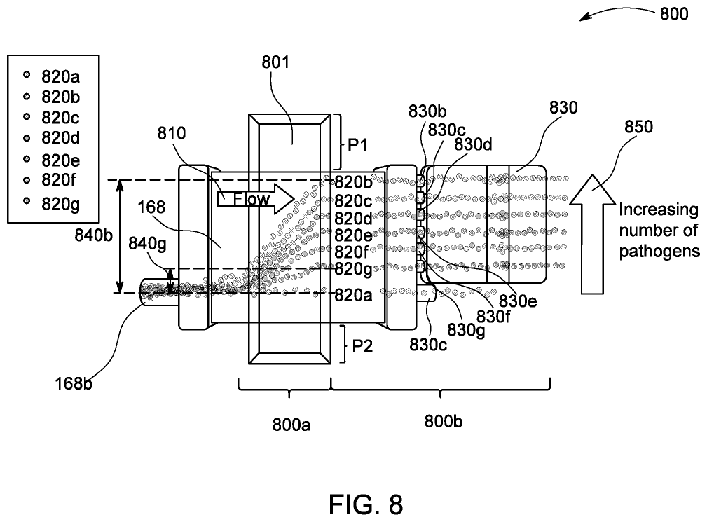

Inset FIG. 2B shows an exemplary channel 168 and magnet 170 configuration 600 located inside portion 150, herein referred to as a “microfluidic magnetic separator.” The separator configuration 600 shown in FIG. 2B is shown in more detail in FIG. 6A and discussed in more detail below. Note that other separator configurations (e.g., configuration 800 discussed below in the context of FIG. 8) may be used in conjunction with system 100 instead of configuration 600 shown in FIG. 2B. Exterior housing 176 may include the aforementioned window (not shown) that allows communication of light between the microfluidic channel 168 of the collection portion 150 and the detection system 172.

Turning back to FIG. 2A, base 110 includes output reporting features 120 and 122. Exemplary output reporter 120 reports “indicators” that may indicate a status of the system 100 (e.g., powered on, ready, internal temperatures, whether clogs or malfunctions exist in the portions 150 or 110, etc.). Output reporter 122 may also indicate a simple positive/negative report. A positive report indicating pathogen infection may be, for example, indicated by illuminating a red LED. A negative report indicating no detection of pathogen infection may be indicated by illuminating a green LED. It is to be understood that the colors and indicators are merely exemplary. Other configurations are possible and within the scope of the present disclosure. For example, a touch screen or other display may be provided.

### Reagents and Labeling Systems

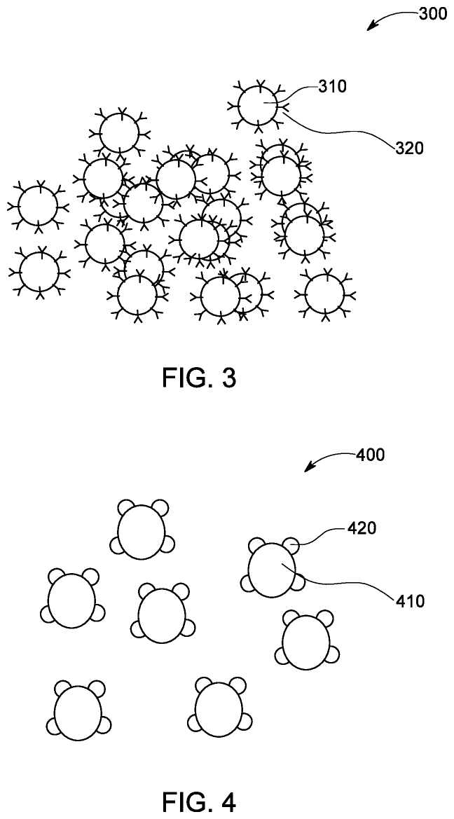

FIG. 3 presents an exemplary pathogen labeling system 300 that may be used in conjunction with the present disclosure and system 100. FIG. 3 shows magnetic particles 310 coated with antibodies 320. The coated magnetic particles 310 may be included among the reagents 164 adjacent to the collection tank 160, as shown in FIG. 1. Patient samples may bond or adhere to the coated magnetic particles 310, as described below, when the sample is introduced to the collection tank 160. As described in more detail below, the means of attachment between the sample and the magnetic particles 310 is coating 320.

Coating 320 may include any relevant antibody, such as an antibody that preferably binds to a known site on a known pathogen that is the subject of the test targeted by system 100. For example, coating 320 may bind to SARS-CoV-2 or other coronavirus. Coating 320 may include a monoclonal antibody directed towards the spike protein or other portion of such coronaviruses, or other pathogens. However, it is to be understood that other antibody-like agents are possible, depending on the application, such as aptamers. In addition, or in alternative, element 320 may include other molecules such as single-chain variable fragments (ScFvs). Antibodies 320 may bind to pathogens other than coronavirus (e.g., tuberculosis or malaria, or various bacteria). Antibodies 320 may be altered, substituted, or changed such that they bind to viruses or other pathogens unknown at the time of writing or otherwise not specifically discussed herein. Although FIG. 3 shows an approximately uniform distribution of coating 320 with respect to magnetic particle 310, it is to be understood that this is merely exemplary. Such a uniform distribution is only one example of system 300 and is not necessary for system 100 to operate as disclosed.

System 300 may include any suitable immunomagnetic label, including magnetic particles 310. Such particles 310 may include, for example, magnetic nanoparticles. Although, in principle, magnetic particles of various sizes may be employed, properties of such particles that may affect attachment (e.g., steric hindrance) should be considered. They may also include Dynabead M450, which is a monodisperse polystyrene bead doped with magnetite. Other exemplary forms of magnetic particle 310 include colloidal magnetic labels such as the MACS particles, which is a dextran particle doped with magnetite. Molecular magnetic labels may also be used. These include ferritin, an iron storage protein. Immunomagnetic labels typically include, for example, a paramagnetic compound or molecule joined to either a primary or secondary antibody. Labeling is performed by attaching the antibody to a marker of interest on the target pathogen (i.e., a pathogen being tested for by system 100). Any suitable magnetic materials may be used for magnetic particle 310, including materials comprising cobalt, iron, nickel, neodymium, magnetite, etc. The magnetic materials may comprise non-magnetic portions (e.g., embedding magnetic materials in a non-magnetic matrix, such as a polymer or glass).

FIG. 4 shows another pathogen labeling system 400 that may be used in conjunction with system 300 in testing system 100. As shown in FIG. 4, system 400 may include fluorescently labeled particles 410 (particles, including “microparticles,” may equivalently be referred to as “microbeads,” as used herein) that are coated with another coating 420, which may include an antibody specific for a different aspect of the testing target pathogen than coating 320. As discussed above in the context of coating 320, coating 420 may include molecules that are not antibodies (e.g., angiotensin converting enzyme 2 (ACE2), aptamters, and ScFvs), or another biomacromolecule capable of binding to a testing target pathogen. Coating 420 may also include an antibody specific for a different epitope of the SARS-CoV-2 or other coronavirus spike protein than coating 320.

Fluorescently labeled particles 410 may be of a number of different sizes. In some variations, particles 410 may be nano or microparticles. In any case, the size of particles 410 should be chosen to balance several practical considerations in the microfluidics and magnetic measurements described below (e.g., whether the induced magnetophoretic mobility due the paramagnetic tag/diploe moment in the field gradient of a magnetic separator/ferrograph configuration is larger than viscous drag forces proportional to the radius of the particle). In cases where magnetic force is small, large drag forces should be avoided. However, particles 410 should also be large enough to be readily observed by detection system 172.

Although not shown in FIG. 4, fluorescently labeled particles 410 are preferably inclusive of fluorescent materials, such as any fluorophores commonly used in immunofluorescence. Fluorescent materials may include fluorescent proteins, non-protein organic fluorophores, as well as certain dyes. Common fluorescent dyes include are Fluorescein isothiocyanate (FITC), Tetramethylrhodamine (TRITC) or Alexa Fluor® dyes. Such florescent materials may enable relatively quick and easy detection of fluorescently labeled particles 410 and pathogens attached to them. Fluorescently labeled particles 410 may comprise glass, polymer, or some other suitable structural material that can accommodate the fluorescent materials. In alternative variations, fluorescently labeled particles 410 include other materials that may be detected by optical, radiological, magnetic, chemical, or electrical means. Moreover, system 400 may include any suitable immunofluorescent label. Immunofluorescent labels typically include, for example, a fluorescent molecule joined to an antibody. System 400 may also incorporate particles 410 that are dyed. This can have the advantage of rendering particles 410 significantly brighter than a fluorophore.

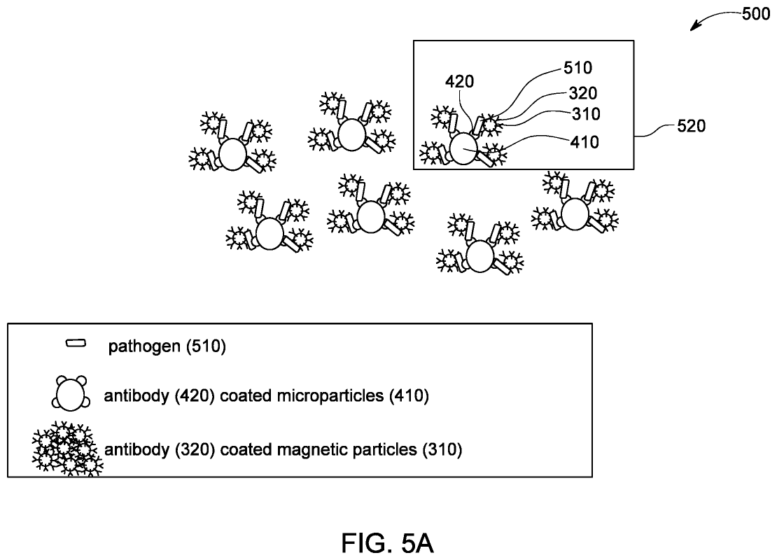

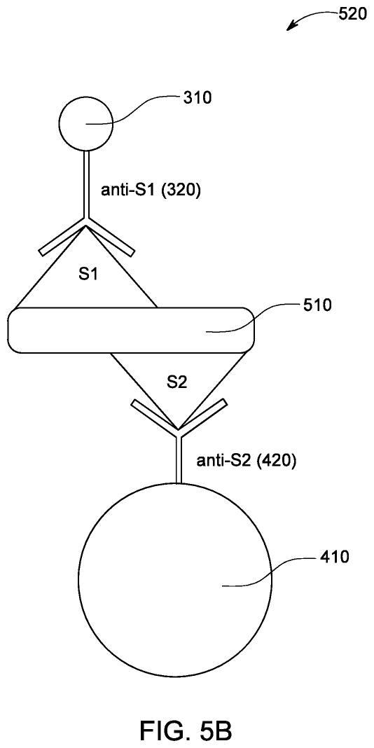

FIG. 5A shows systems 300 and 400 combined in system 500 for labeling testing targeted pathogens 510. In system 500, pathogen 510 is bound both to coating 320 and coating 420 to create an aggregate 520. Note that the term “aggregate” is used herein to refer to an agglomeration of particles. The aggregate itself may be, consistent with this disclosure, also called a “particle.” As mentioned above, antibodies 320 and 420 may be chosen so that magnetic particle 310 and fluorescently labeled particle 410 bind to different subunits of the same pathogenic protein. FIG. 5B illustrates this case. More specifically, FIG. 5B shows a schematic of agglomerate 520 where the magnetic particle 310 and the fluorescent labeled particle 410 are bound to different subunits S1 and S2, respectively, of a spike protein on pathogen 510, where pathogen 510 is a coronavirus. Such antibodies are currently commercially available for SARS-CoV-2. Since the SARS-CoV-2 S protein is known to share ˜76% homology with SARS-CoV S protein and a lower homology with the S proteins from other common coronaviruses, it is important to develop an antibody testing system that can recognize the target protein with high affinities and minimal undesired cross-relativities.

Aggregates 520 of magnetically tagged pathogens 510 and fluorescently labeled particles 410 will form in suspension (e.g., inside collection tank 160), creating a magnetic dipole for each aggregate 520. This binding provides pathogen 510 with potentially both a magnetic marker (310) and a fluorescent marker (410), facilitating two complimentary means of detection or differentiation (i.e., magnetic and fluorescence). This labeling complementarity can be advantageous. For example, it can provide amplification of the relatively low level of pathogen signal one expects in a breath sample. This is because using a sandwich assay (e.g., system 500) binding a fluorescently labeled particle 410 to a pathogen 510 along with a magnetic particle 310, can allow multiple site binding by either particle 310 or 410. In other words, each pathogen 510 can be labeled with multiple markers effectively doubling or tripling (or more) the magnetic effect on a single pathogen 510 caused by a magnetic field. This increases the magnetophoretic mobility for displacement type separations and attachment force for binding separations. Studies have shown that breath droplets contain a relatively low level of pathogens (e.g., SARS-CoV-2) than samples derived from other, more invasive methods (e.g., swabbing). The aforementioned two-stage enrichment process (i.e., first tagging the pathogen with a magnetic particle 310, then tagging the magnetically tagged pathogen with a fluorescently labeled particle 410) results in aggregates 520 of magnetically and fluorescently labeled pathogen in suspension.

Turning back to FIG. 5A, although aggregates 520 are represented as being identical, it is to be understood that this is not always the case. Aggregates 520 may differ in terms of an amount of pathogen 510 attached, fluorescently labeled particles 410, and importantly in the number of adhered magnetic particles 310. In particular, differences in magnetic particle 310 adherence to pathogens 510 can create aggregates 520 with different magnetic dipole moments. As discussed in more detail below, these differences in magnetic dipole moments can be used to separate different particles and estimate quantities such as pathogen load.

### Magnetic Separation and Analysis

Basic principles of magnetic particle separation are discussed in greater detail in U.S. Pat. No. 5,968,820 to Zborowski et al., herein incorporated by reference in its entirety. Briefly, a flow of magnetically labeled particles (e.g., magnetic particles 310 or aggregates 520) in a conduit such as microfluidic channel 168 will change direction in response to a varying magnetic field within the channel 168. In contrast, a flow of non-magnetic particles will be unaltered by variations in magnetic field. Therefore, introducing magnetic field variations in microfluidic channel 168 can differentiate magnetically labeled particles, or aggregates, from particles without magnetic labels. In addition, flow changes in response to magnetic field variation can provide an understanding of the amount of pathogen attached to an aggregate 520. Flow of magnetically tagged particles (aggregates 520) will be dependent on VB², which is the gradient of the magnetic flux density to the second power, weighted by, among other things, the density of magnetic particles attached (a). See U.S. Pat. No. 5,968,820 at Equations 1 and 2. In the case of system 500, density of magnetic particles attached (a) of an aggregate 520 depends on the quantity of magnetic particles 310 bound to its surface via coating 420 (and pathogen 510). Therefore, passing a magnetically labeled patient sample through a varying magnetic field can allow separation of aggregates 520 based on their magnetic attachment density. Since, in this context, the number of attached magnetic particles depends on pathogen 510 attachment, this separation of particles also relates to the number of pathogens attached.

In principle, any suitable configuration that exploits the magnetic particle attachment density effect (hereinafter referred to as dipole moment) for sorting particles 520 can be used to detect pathogens with system 100. That is, any of the dipole separator systems disclosed in U.S. Pat. No. 5,968,820 (e.g., system 200 or the dipolar fractional sorter of FIG. 4) could be employed in system 100. Descriptions of several specific systems that may be used in conjunction with testing system 100 follow. However, it is to be understood the systems described herein do not represent an exclusive or exhaustive list of such systems that may be used with testing system 100. Testing system 100 and the present disclosure can be used with any suitable magnetic separation system whether disclosed explicitly herein, disclosed herein by reference, or not.

FIG. 6A shows a schematic of the interior of the microfluidic channel 168 and magnet 170 portion of system 100 in one exemplary variation 600. The configuration shown in FIG. 6A is generally referred to as High Gradient Magnetic Separation (HGMS) and can be referred to as a “ferrograph” or “microferrograph.” In it, the magnet 170 creates a several regions of high magnetic field gradient in the channel 168 that tend to trap aggregates 520 based on their magnetic properties.

Microfluidic channel 168 and magnet 170 together form microfluidic magnetic separator 600. As shown in FIG. 6A, channel 168 has been fabricated (e.g., via microfabrication or MEMS) inside an enclosed microchannel assembly 610. The channel 168 may be pressed against the interpolar gap of magnet 170. The magnet 170 may include a magnetic manifold comprising permanent (e.g., cobalt, iron, rare earth, NdFeB) or other magnets. In the example in FIG. 6A, the magnet 170 comprises four trapezoidal magnetic elements 170a-170d placed in series or in an array. The magnetic elements 170a-170d may be adjoined to one another using a non-magnetic material 620 (e.g., a steel, such as a low carbon steel). In this configuration, the smaller of the two parallel sides of the trapezoid form the interpolar magnetic gaps N/S, as shown in the case of magnetic element 170b. Element 630 is an optional base on which the magnet 170 rests. Element 630 may be, for example, part of the exterior housing 176 shown in FIG. 2.

In order to maximize a magnetic field gradient provided by magnet 170, the magnetic elements 170a-170b may be positioned with alternating polarity (i.e., 170a N/S as shown in FIG. 6A, 170b S/N, 170c N/S, and 170c S/N). The dimensions of the channel 168 and magnet may vary and should be set according to the particular application (e.g., according to the types, weights, and volumes of particles/aggregates 310/410/520 used and their magnetic dipole moments, as well as anticipated pathogenic attachment on aggregates 520, and the properties (e.g., viscosity) of a carrier fluid). An exemplary thickness of the channel 168 walls is 400 μm and diameter 250 μm. The height and widths of the magnetic elements 170a-170d may be, for example, on order of mm. In this configuration, an example magnetic gap width (i.e., distance between N and S in magnetic element 170a) may be on order of 1 mm.

FIG. 6A also shows a schematic of detection system 172 in an exemplary placement adjacent to channel 168. Detection system 172 can provide light to cause particles 410 (and, therefore, aggregates 520) to fluoresce. If detection system 172 is located on the base 110, it may communicate light through the aforementioned window in the exterior housing 176 of the collection portion 150. System 172 can also resolve detected fluorescent light vs. distance along channel 168. In this case, detected fluorescence correlates to a number of aggregates 520. As discussed in more detail below, a correlation of fluorescence with distance along the channel can reveal the viral load in the patient who provided the sample, among other things. The microchannel assembly 610 may be at last partially transparent, allowing light from fluorescence of particles 410 in the channel to reach the detection system 172. The entire microchannel assembly 610 may be transparent. Alternatively, a portion of the assembly 610 (e.g., the portion adjacent to the detection system 172 or window described above) may be transparent.

FIG. 6B shows a photograph of an actual implementation of the microfluidic channel 168 and magnet 170 of FIG. 6B as microfluidic magnetic separator 650. A penny 601 is included for perspective. Dimensions of the components in microfluidic magnetic separator 650 correspond to the exemplary dimensions discussed in the context of microfluidic magnetic separator 600 above.

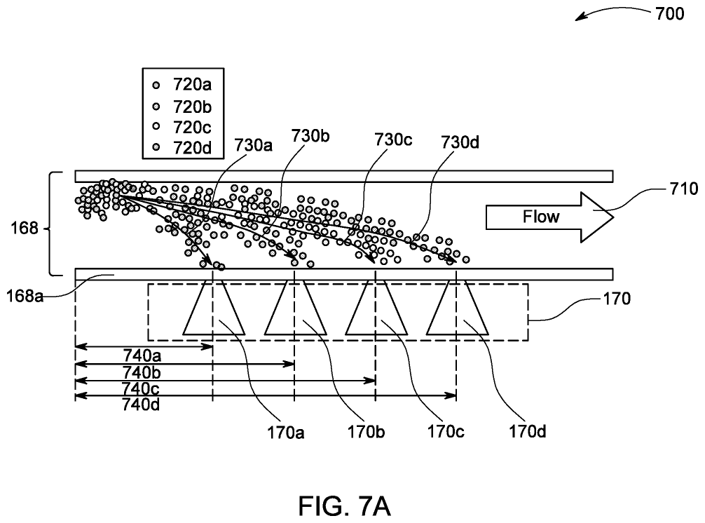

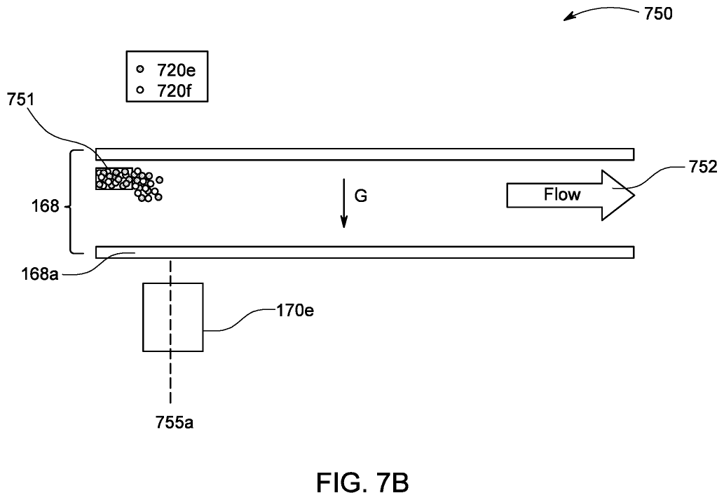

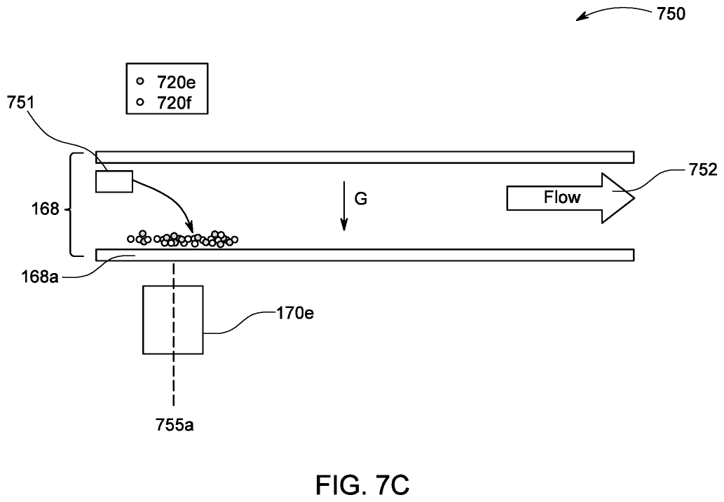

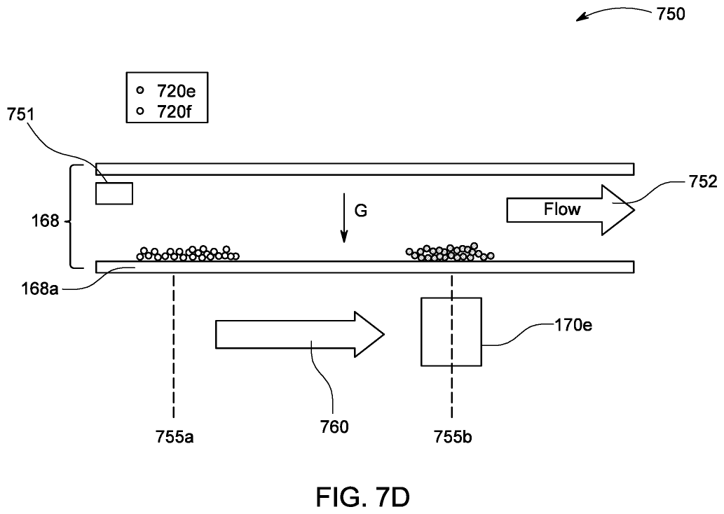

FIG. 7A shows a schematic of the operation of an exemplary HGMS microfluidic magnetic separator configuration 700 (e.g., implemented via microfluidic magnetic separator 600 or 650) in conjunction with system 100. As shown in FIG. 7A, microfluidic magnetic separator 700 includes magnet array 170 and microfluidic channel 168 (as also shown in FIG. 1 for system 100). The orientation of aggregate flow 710 in FIG. 7A, as well as the orientation of microfluidic channel 168, has been rotated from the orientation of flow 171 and the microfluidic channel 168 in FIG. 1. This is merely for illustrative purposes and does not imply a difference in design or configuration.

As discussed above in the context of FIG. 1, the direction of Flow 710 in channel 168 can be reversed (e.g., by withdrawing plunger 152). Flow 710 can be cycled from (e.g., flow forward, then reverse, flow forward, then reverse, etc.) a number of times. As discussed in the context of FIG. 1, such cycling can be useful to help sample accumulate on the microfluidic magnetic separator 700, to mix with carrier fluid, and to increase signal to noise ratio. Although not shown in FIG. 7A, a collection tank may be placed at one or both ends of channel 168 (or anywhere else on channel 168) to collect sample for cyclic flow or for storage. These principles of cyclic flow, reversing flow direction, and fluid storage may be applied to any of the variations described herein.

For illustration purposes, FIG. 7A shows four different types of aggregates 520 (FIG. 5A), i.e., aggregate types 720a-720d, that differ in terms of magnetic dipole moment. Aggregates 720a-720d are aggregates 520 formed from forcing a patient sample (taken via sample extractor 154) into contact with reagents 164 in the collection tank 160 of sample collection portion 150. Use of multiple magnetic elements (e.g., four as in FIG. 7A) may increase collection efficiency of aggregates 720a-720d because employing more elements gives aggregates more time to deposit. The flow rate of aggregates 720a-720d in channel 168 can be increased, in some cases, without loss of sensitivity. It is to be understood that any suitable number of magnetic elements may be used in magnet 720 and still be consistent with this disclosure. In some cases, only a single magnetic element may be used.

While flowing through microfluidic channel 168, aggregates 720a-720d are subject to a varying magnetic field B emanating from magnetic elements 170a-170d. They are attracted to the elements 170a-170d and trapped onto a stationary surface 168a of the channel 168. Since the attraction depends on dipole moment of the aggregate, the trapping effectively sorts aggregates 720a-720d according to magnetic dipole moment. After the aggregates 520 have been trapped, data can be collected and/or analyzed (e.g., by measuring the fluorescence of fluorescently labeled particles 410 within the aggregates 520).

Aggregates 720a have the highest magnetic dipole moment and are colored black. Aggregates 720a may have the highest magnetic dipole moment because they have a relatively large number of attached magnetic particles 310, correlating to a relatively high number of attached pathogens 510. Aggregates with the lowest magnetic dipole moment are labeled 720d and are colored orange. Aggregates 720d may have the lowest magnetic dipole moment because they have a relatively small number of attached magnetic particles 310 due to a relatively low number of pathogens attached. Aggregates with intermediate magnetic dipole moment are labeled 720b and 720c and are colored brown and green, respectively. The different magnetic dipole moments of aggregates 720a-720d correlates to differing numbers of magnetic particles 310 attached to each aggregate, as discussed above, due to differing numbers of attached pathogens.

As shown in FIG. 7A, aggregates 720a with the highest magnetic dipole moment traverse path 730a, ultimately collecting near magnetic element 170a. Aggregates 720a traverse the smallest average distance 740a from the start S of the microfluidic channel 168. As discussed above, these aggregates may have a higher dipole moment because they have increased attachment of magnetic particles 310 due to higher number of attached pathogens 510. In contrast, aggregates 720d traverse the greatest distance 740d from the start S of the microfluidic channel 168 along path 730d. This is because aggregates 720d have the lowest magnetic dipole moment, which may be due to fewer attachments with magnetic particles 310. Intermediate aggregates 720b and 720c may travel intermediate distances 730b and 730c, respectively, due to intermediate levels of magnetic particle 310 attachment and intermediate levels of pathogenic attachment.

It is to be understood that, though magnetic elements 170a-170d have been illustrated in FIG. 6 as identical, they need not be. Magnetic elements 170a-170d may differ in terms of magnetic field origin (e.g., permanent magnets or electromagnets), field strength, and/or other properties. In addition, although four magnetic elements are shown, other suitable numbers of magnetic elements may be used. Some cases may require only one magnetic element.

Once aggregates 720a-720d have collected near a magnetic element, they have effectively been sorted. Their relative distribution may then be assessed using optical or other detection techniques. As discussed above, one such technique is to collect fluorescence data from fluorescently labeled particles 410 in aggregates 520 at various positions along microfluidic channel 168. A higher concentration of aggregates at a shorter distance from the channel start S (e.g., 740a or 740b) may indicate higher pathogen load in the patient. A higher concentration of aggregates at a longer distance from the channel start S (e.g., 740d or 740c) may indicate lower pathogen load in the patient. Fluorescence of the fluorescently labeled particles 410 in aggregates 720a-720d can be excited by any suitable means (e.g., by using detection system 172 in FIGS. 1 and 6A), such as by using LED light. However, LED light is merely exemplary. Other illumination sources may be used in the context of the present disclosure to, for example, cause fluorescently labeled particles 410 to fluoresce. For example, fluorescence data may be obtained by illuminating fluorescently labeled particles 410 using UV radiation, laser light, and/or any other suitable type of radiation.

Fluorescent intensity at various positions in the channel 168 may be collected via cameras (e.g., for digital image processing), light sensors, or any other suitable means, including, but not limited to, sensing modalities such as giant magneto resistance, hall sensors, resonant sensors and impedance. It may be advantageous to obtain digital images of the collected aggregates for further analysis of their distribution within a region close to one or more of the magnetic elements 170a-170d. For example, the data may reveal whether aggregates are monodisperse in terms of their attachment to fluorescently labeled particles 410. This data may also be used for system diagnostics or fine tuning of the above-described equipment and processes (e.g., to determine whether magnetic field variation over regions of channel 168 creates variations within that region). Alternatively, each collection region may be assigned a single average brightness based on the collected fluorescent intensity. This latter approach may be useful in version of system 100 in which one or more portions (e.g., collection portion 150) is simplified and/or disposable. Single valued, average fluorescence readings may also allow for rapid positive/negative testing.

Once fluorescence intensity data is obtained, it may be collected and analyzed by system 100 (e.g., by the electronics 118 included in the base 110). For example, digital image processing may be performed on fluorescence intensity images, as described above. The digital image processing may be performed by any suitable software platform, e.g., Image Pro Plus. Examples of image processing that may be used include edge-analysis for differentiating individual aggregates/particles/cells/other collected objects, filtering to remove noise and threshold image intensity (e.g., to remove debris and large objects not consistent with aggregate or particle size) and generating binary maps of aggregates to isolate them from the background. The latter technique may facilitate better quantification of aggregates, e.g., more accurate aggregate counting via aggregate counting algorithms. It may further enhance data accuracy by identifying objects in the images that are too large or too small to be counted as aggregates under examination (e.g., debris).

FIGS. 7B-7D show another magnetic separation configuration 750. In configuration 750, there are at least two types of aggregates 720e and 720f that differ in terms of magnetic properties. The first type, aggregates 720e, have a substantial magnetic dipole moment because they have a substantial number of attached magnetic particles 310 (e.g., like aggregates 520 in FIG. 5A). This likely indicates that aggregates 720e have substantial pathogen 510 attachment (see FIG. 5A). Aggregates 720e are colored orange. The second type, aggregates 720f, do not have a substantial dipole moment because they lack a substantial number of attached magnetic particles 310. Aggregates 720f are colored blue. Aggregates 720f likely have a lower pathogen 510 content than aggregates 720e.

As shown in FIG. 7B, both aggregates 720e and 720f are introduced to channel 168 together at 751. Generally, both aggregates 720e and 720f flow in the direction of 752 down channel 168. Flow 752 may be actuated by movement of plunger 152 (FIG. 1). However, aggregates 720e and 720f may also settle on channel surface 168a due to interaction with forces acting on them in the channel. These acting forces may include gravity G, as shown in FIG. 7B. They may further include an effective attraction to magnet 170e experienced by aggregates 720e via their dipole moments. In this arrangement, both G and magnetic interactions tend to pull the aggregates towards surface 168a. Magnet 170e may have any suitable form, including the trapezoidal form of magnetic elements 170a-170d in FIG. 7A.

As shown in FIG. 7C, both aggregates 720e and 720f follow an average path 753 from entrance 751 to the vicinity of magnet 170e. The exact path followed by aggregates 720e and 720f may be somewhat different since they may experience different forces. In any case, as shown in FIG. 7C, both aggregates 720e and 720f settle on channel surface 168a near magnet 170e at position 755a.

As shown in FIG. 7D, magnet 170e may then be moved along direction 760 to a second position 755b. This movement of magnet 170e may induce flow of magnetic aggregates 720e along direction 760. Aggregates 720e will tend to move with magnet 170e because of the their effective attraction it via their dipole moments. Aggregates 720e will re-settle on channel surface 168a at the new position of magnet 170e (i.e., 755b). On the other hand, aggregates 720f remain at position 755a despite the movement of magnet 170e. This is because aggregates 720f lack a substantial dipole moment and, therefore, lack an effective attraction to magnet 170e.

As discussed above in the context of FIG. 5, aggregates 720e have a substantial dipole moment because they are attached to magnetic particles 310 via a pathogen 510. Therefore, movement of aggregates 720e in response to their dipole interaction with magnet 170e (FIG. 7D) effectively differentiates them from aggregates 720f based on pathogen 510 attachment. In other words, response to movement of magnet 170e effectively separates or sorts aggregates with a substantial pathogen attachment (i.e., magnetic aggregates 720e) from those without substantial pathogen attachment (i.e., non-magnetic aggregates 720f). This separation can be used in conjunction with fluorescence detection (e.g., using detection system 172) to detect whether a patient tests positive for pathogen 510. As described above, such separation of aggregates can also be used to estimate pathogen 510 load in the patient. Any of the detection methods described above in context with configuration 700 may also be used with configuration 750.

FIG. 8 shows a schematic of the operation of another exemplary microfluidic magnetic separator configuration 800 (e.g., implemented via microfluidic magnetic separator 600 or 650) in conjunction with system 100. The configuration 800 is generally referred to as Open Gradient Magnetic Separation (OGMS), in contrast to the HGMS configurations 600, 650, 700, and 750. As shown in FIG. 8, microfluidic magnetic separator 800 includes magnet 801 and microfluidic channel 168 shown in FIG. 1. One exemplary advantage of configuration 800 is that it could potentially be more readily used to determine levels of virus expression rather than a binary (positive or negative).

Magnet 801 in FIG. 8 differs from the magnet 170 in the HGMS configurations (600, 650, 700, and 750) in that magnet 801 does not include discreet magnetic elements, each with unique poles (e.g., 170a-170d; see, e.g., FIG. 6) to impart regions of high field gradient. Instead, magnet 801 is positioned so that its two poles (N/S), labeled P1 and P2, are on either side of channel 168. This way, magnet 801 provides a moderate magnetic field gradient throughout channel 168, or an isodynamic field. As discussed in more detail below, the moderate field gradient imparted by magnet 801 causes deflection of flowing aggregates 520 in the stream rather than trapping them on a surface 168b of the channel 168 (e.g., as shown in FIG. 7A). The amount of deflection is a function of the aggregate's 520 magnetic dipole moment and its geometry. One advantage of configuration 800 is that more aggregates 520 may be collected this way for an enhanced signal. Aggregates can be sorted by magnetophoretic mobility which is related to the amount of pathogen 510. This could be used to determine total dose per breath of the patient (assuming a single breath sample) and an infectivity associated with the patient. This data can be collected, measured and released for subsequent analysis such as genomic analysis.

The placement of the magnet 801 in configuration 800 creates a region 800a in which aggregates are subject to changing magnetic flux. There is another region of configuration 800, collection region 800b, in which the aggregates 820a-820g are shielded from changing magnetic flux. The latter will be discussed in more detail below.

As in configuration 700, the orientation of flow 810 shown in FIG. 8 is rotated with respect to the orientation of flow 171 in FIG. 1. This is merely for illustrative purposes and does not imply a difference in design or configuration. Both configurations 700 and 800 can be used in conjunction with system 100. Their channels can be oriented with respect to portion 150 as shown in FIG. 2B, i.e., with aggregate flow in the direction of arrow 171.

For illustration purposes, FIG. 8 shows seven different types of aggregates 520 (FIG. 5A), i.e., aggregate types 820a-820g. Aggregates 820a-820g differ in terms of magnetic dipole moment. They are aggregates 520 formed from forcing a patient sample (e.g., a breath sample taken via sample extractor 154) into contact with reagents 164 in the collection tank 160 of sample collection portion 150. Aggregates 820a-820g enter the channel 168 via channel intake 168a. However, the way the aggregates 820a-820g exit channel 168 depends on how magnet 170 affects their trajectory in the channel.

Orange aggregates 820a having the lowest magnetic dipole moment are virtually unaffected by magnet 170 over the entire region 800a. They exit channel 168 through port 168c without substantial deflection (i.e., following an essentially straight path across channel 168). In this configuration, aggregates 820a collected or measured via port 168 may be considered negative with respect to the target pathogen of the test. This is because they have collected too few magnetic particles 310 to be affected by the microfluidic magnetic separator test implemented by configuration 800, implying that they have little to no pathogen 510 adhesion. The trajectories of all other aggregates 820b-820g in channel 168 are affected to some degree and, therefore, represent positive results. They are positive in the sense that their deflections indicate that aggregates 820b-820g have a significant magnetic dipole moment, correlating to a significant pathogenic attachment.

In collection region 800b, aggregates 820b-820g are separated and collected according to their various deflections. More specifically, positive aggregates 820b-820g are directed to one of the channels 830b-830g in the analyzer 830 (note that channel label 830a has been omitted for convenience, so that the alphabetic portion of the channel labels matches the aggregate labels). As shown in FIG. 8, for example, aggregates 800b are collected via output channel 830b which separates them from other aggregates in the analyzer 830. Similarly, aggregates 820c-820g are separated and channeled into their respective output channels 830c-830g. Although not indicated as an output channel in analyzer 830, output 168g, which collects pathogenic negative aggregates 820a, may also be fed to the analyzer 830 for analysis.

Once aggregates 820a-820g have been magnetically sorted according to configuration 800 or 750, their relative distribution may be assessed using fluorescence techniques, as described in detail in the context of configuration 700. Any of the techniques discussed above in the context of configuration 700 or 750 may be used to collect and analyze data from configuration 800.

### Methods of Operation

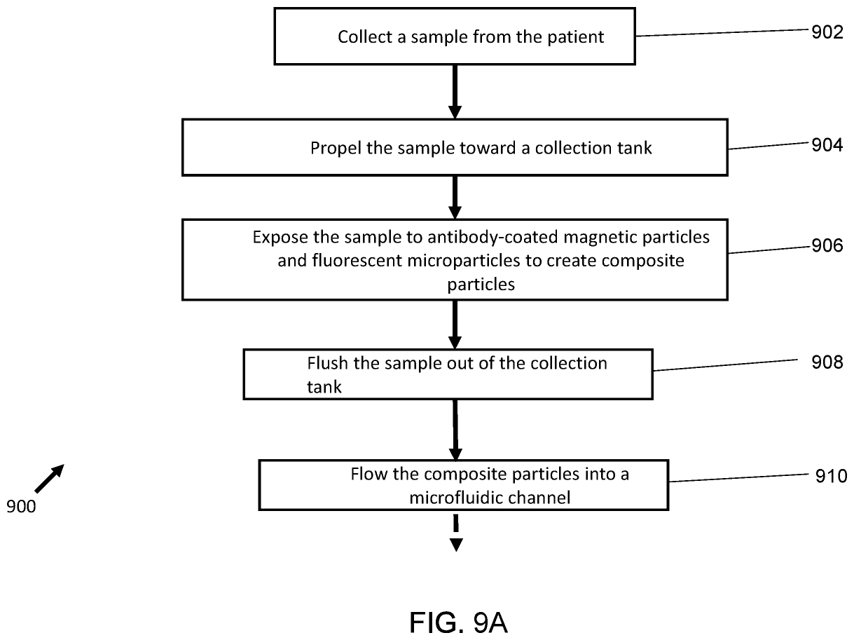

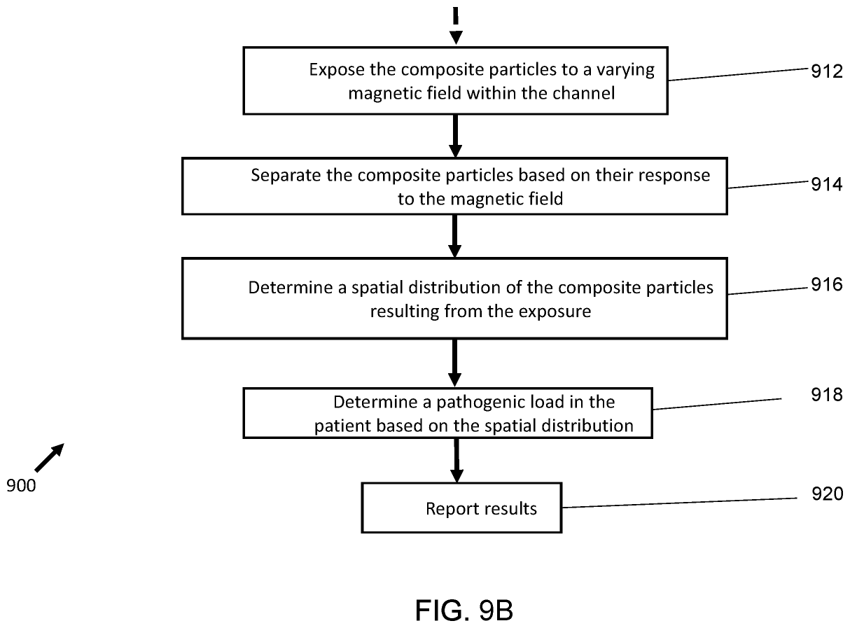

FIGS. 9A and 9B present a flowchart showing an exemplary method for using system 100 to detect a potential pathogen.

Starting with FIG. 9A, in step 902, sample collection portion 150 is used to collect a sample from the patient. As discussed above, the sample can be collected in a variety of ways, including by using sample extractor 154 as a mouthpiece. In this case, the patient may blow his or her breath into the sample extractor 154 so that the breath enters inlet tube 156. The patient's breath may contain the target pathogen, as well as debris that may later be removed by filtering.

In step 904, the sample is propelled downward through inlet tube 156 to the collection tank 160. This may be accomplished by gravity. In exemplary cases, an interior of the inlet tube 156 will be hydrophobic so that the sample (typically comprising water) will not stick to the interior. Instead, the sample will settle towards the bottom of the tube 156 where the collection tank 160 may be located (FIG. 1). In certain cases, the sample may be propelled down the inlet tube 156 via emission device 162. For example, emission device 162 may emit a saline solution (e.g., as a spray) to wash the sample down inlet tube 156 towards collection tank 160. In this step, the sample may also be filtered to remove debris.

In step 906, the sample is in collection tank 160. While in the tank 160, the sample may be exposed to magnetic particles 310 and fluorescently labeled particles 410. The magnetic particles 310 bind to pathogens 510 in the sample via coating 320. The fluorescently labeled particles 410 bind to pathogens 510 in the sample via coating 420. As discussed above, antibodies 320 and 420 may be selected to bind to different sites on the pathogen 510. For example, in the case that the pathogen is SARS-CoV-2, antibodies 320 and 420 may bind to different epitopes of the SARS-CoV-2 spike protein. Coating 420 may comprise ACE2 for this purpose. An additional benefit to using ACE2 is that it overcomes the problem noted in the literature that many antibody tests of involving SARS-CoV-2 cross-react with other SARS-type viruses. In other variations, two monoclonal antibodies binding to different parts of an S protein of a coronavirus may be used. The latter variation may be advantageous under certain circumstances because monoclonal antibody affinity may be higher than ACE2 affinity to the S protein of coronaviruses. In any case, binding to the different sites of the pathogen in the collection tank 160 may create aggregate 520, which comprises both magnetic particles 310 and fluorescently labeled particles 410. Therefore, the binding labels the pathogen 510 both magnetically and fluorescently. In the case of greater concentrations of pathogen, aggregates 520 are likely to have an increased binding to the magnetic particles 310 via pathogen 510 and, therefore, a greater magnetic dipole moment. This quality allows magnetic separation, as discussed in more detail in the context of steps 912 and 914 below.

In step 908, the sample may be flushed out of the collection tank 160. Although not shown in the figures, there may be a valve to stop reagents 164 from entering either outlet channel 158 or microfluidic channel 168. One means of flushing uses plunger 152 to physically push the sample. Plunger 152 may be actuated by a piston 116 located in the interface portion 112 of the base 110. A motor or actuator 114 on the base 110 may actuate the piston 116 to actuate the plunger 152. This action may also push uncaptured portions of the sample into outlet tube 158 and through filter 166. It may further cause the uncaptured portions to be expelled from portion 150 through filter 166. Filter 166 may remove pathogens from the uncaptured portions so that the pathogens are not emitted into the ambient environment.

In step 910, the sample may be flowed into microfluidic channel 168, which may be part of the microfluidic magnetic separator (e.g., as shown in FIGS. 2B, 6A, and 6B). The motion of the sample from the outlet tube 158 into the microfluidic channel 168 may be actuated by the motion of plunger 152. As part of this motion, the uncaptured part of the sample may be forced through exhaust filter 166 before exiting collection portion 150 to the ambient environment.

Turning to FIG. 9B, in step 912 the aggregates 520 of the sample are exposed to a varying magnetic field within the microfluidic channel 168. The exposure setup may conform to any suitable configuration of a microfluidic magnetic separator discussed herein (e.g., 600, 650, 700, 750, or 800), as well as other configurations of microfluidic separators incorporated herein by reference. In addition, other forms of known microfluidic separators not explicitly incorporated in this disclosure are within the scope of this disclosure and may be used with system 100.

In step 914, the exposure to varying magnetic fields (step 912) should separate the aggregates 520 based on their magnetic dipole moments. Since the magnetic dipole moments of aggregates 520 depends on the attachment of magnetic particles 310 via pathogens 510, separation should also reflect an amount of pathogen 510 attached to the aggregates 520. In this step, the separated aggregate 520 may collect at different portions of the channel 168 based on their dipole moments, as shown in FIGS. 7A and 7D. They may also be directed into different channels (e.g., channels 830b-830g in FIG. 8) based on their magnetic deflection. It is to be understood that other suitable methods of separating the aggregate 520 are possible and within the scope of this disclosure.

In step 916, a spatial distribution of the aggregates 520 in the microfluidic channel 168 is then determined. This spatial distribution can be assessed in a variety of ways. For example, the spatial distribution can be obtained by using fluorescence of the fluorescently labeled particle 410 portion, as described above. Briefly, a detection system (LED, UV, or otherwise) may illuminate the aggregate 520, causing them to fluoresce. The fluorescence may be detected, for example, via a detection system 172 located on the collection portion 150. It may also be detected by a detection system (not shown) located on the base 110. In either case, the detection system may include cameras and/or photodetectors positioned so that they can record fluorescence throughout the spatial distribution of aggregate 520 in channel 168. Another means of determining the spatial distribution of aggregate 520 is simply by counting the aggregates in variously spaced collection channels (e.g., channels 830b-830g in FIG. 8). Any suitable method of aggregate 520 counting may be used, including measuring aggregate count via fluorescence, as described above. Other examples include using a Coulter counter.

In step 918, a pathogenic load in the patient (e.g., a viral load, in the case the pathogen is a virus) may be assessed based on the spatial distribution of the aggregate 520 in channel 168, as assessed in step 916. Since the spatial distribution correlates with pathogen 510 attachment, as discussed above, it may be understood to indicate pathogen load. For example, a greater concentration of aggregate 520 in the vicinity of magnetic element 170a (FIGS. 7A and 7D), may indicate a higher pathogen load. This is because the result would indicate a higher degree of magnetic dipole moment in the aggregate 520, corresponding to a higher degree of pathogen 510 attachment. Similarly, a greater concentration or count of aggregate 520 in channel 820b (FIG. 8) would indicate a greater deflection and dipole moment, therefore a greater degree of pathogen 510 attachment. This could also correlate to higher pathogen load in the patient.

In certain cases, a threshold pathogen load could indicate a positive result. For example, in some cases, if 50% or more aggregates 520 are detected a distance indicating highest pathogen load (distance 740a in FIG. 7A, distance 755b in FIG. 7D, or distance 840d in FIG. 8), this may indicate that the patient tests positive for the pathogen. Otherwise, the patient tests negative for the pathogen. The threshold may vary according to pathogen and other aspects of experimental setup (e.g., the features of particles 320 and 420, the strength of magnets 170 and 801, etc.). Thresholding could also be a more complicated function of the distribution of particles on surface 168a or in channels 820b-820g. It is to be understood that any such suitable threshold is within the scope of this disclosure. Specific quantifiable thresholds may vary with the experimental specifics. Relevant parameters include the ferrograph configuration (e.g., configurations 700, 750, or 800), dimensions of channel 168, and where in channel 168 the aggregates 520 are injected. A balance between effective magnetic attracting force and other forces (e.g, sheer stress related to particle size relative to channel 168 dimension) may also be important.

In step 920, the results of the testing are reported. Any suitable method of reporting results may be used. For example, the base 110 may report results via a simple display (LED as in element 122 of FIG. 2A, or otherwise) indicating that the patient tests positive or negative for the pathogen 510 based on the determined viral load. Raw or processed data (e.g., fluorescence data, image data, processed image data) may also be reported by any suitable means. Suitable means include reporting via Ethernet, Bluetooth, WiFi, mobile phone network, magnetic disk, flash drive, or other storage media.

While various inventive aspects, concepts and features of the inventions may be described and illustrated herein as embodied in combination in the exemplary embodiments, these various aspects, concepts and features may be used in many alternative embodiments, either individually or in various combinations and sub-combinations thereof. Unless expressly excluded herein all such combinations and sub-combinations are intended to be within the scope of the present inventions. Still further, while various alternative embodiments as to the various aspects, concepts and features of the inventions—such as alternative materials, structures, configurations, methods, circuits, devices and components, software, hardware, control logic, alternatives as to form, fit and function, and so on—may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure, however, such values and ranges are not to be construed in a limiting sense and are intended to be critical values or ranges only if so expressly stated. Still further, exemplary or representative values and ranges may be included to assist in understanding the present disclosure, however, such values and ranges are not to be construed in a limiting sense and are intended to be critical values or ranges only if so expressly stated. Parameters identified as “approximate” or “about” a specified value are intended to include both the specified value and values within 10% of the specified value, unless expressly stated otherwise. Further, it is to be understood that the drawings accompanying the present application may, but need not, be to scale, and therefore may be understood as teaching various ratios and proportions evident in the drawings. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention, the inventions instead being set forth in the appended claims. Descriptions of exemplary methods or processes are not limited to inclusion of all steps as being required in all cases, nor is the order that the steps are presented to be construed as required or necessary unless expressly so stated.

We claim:

1. A method for detecting viral or bacteriological pathogens comprising:
collecting a potentially pathogenic sample via a collector;
binding a first portion of the potentially pathogenic sample to a magnetic particle via a first coating on the magnetic particle;
binding a second portion of the potentially pathogenic sample to a fluorescently labeled particle via a second coating on the fluorescently labeled particle to create aggregates comprising the potentially pathogenic sample, magnetic particle, and the fluorescently labeled particle;
separating the aggregates magnetically;
detecting a fluorescence of the separated aggregates; and
estimating an amount of the pathogen based on the detected fluorescence.

2. The method of claim 1, wherein at least one of:
the collector is a breathalyzer;
the collecting comprises obtaining the potentially pathogenic sample from a patient's breath via the breathalyzer;
the pathogen is a coronavirus;
the coronavirus is SARS-CoV-2;
the first portion of SARS-CoV-2 to which the first coating binds and the second portion of SARS-CoV-2 to which the second coating binds are different epitopes of a SARS-CoV-2 spike protein;
the second coating comprises angiotensin converting enzyme 2 (ACE2); and
the separating comprises separating the aggregates via a microfluidic magnetic separator according to their magnetic dipole moments.

3. The method of claim 2, wherein the separating is according to at least one of:
distances traveled by the aggregates in the microfluidic magnetic separator;
times of travel of the aggregates in the microfluidic magnetic separator; and
flow of the aggregates in the microfluidic magnetic separator.

4. The method of claim 3, wherein the estimating an amount of the pathogen based on the detected fluorescence comprises estimating the amount based on a spatial distribution of the detected fluorescence in the microfluidic magnetic separator.

5. The method of claim 4, wherein the spatial distribution of the detected fluorescence results from the separating.

6. The method of claim 1, wherein the detecting a fluorescence of the separated aggregates comprises:
illuminating the aggregates; and
detecting an amount of fluorescence of the fluorescently labeled particles excited by the illumination.

7. The method of claim 6, wherein the florescence is a fluorescence of the fluorescently labeled particles in the aggregates.

8. The method of claim 1, further comprising estimating a viral load in the patient based on the estimated amount of pathogen.

9. A device for detecting viral or bacteriological respiratory pathogens comprising:
a breath capture portion for capturing a potentially pathogenic sample comprising:
a mouthpiece;
an inlet tube;
a collection tank connected to the mouthpiece via the inlet tube, the collection tank comprising:
magnetic particles coated with a first coating that binds to a first portion of a pathogen; and
fluorescently labeled particles coated with a second coating that binds to a second portion of the pathogen; and
an outlet tube connecting the collection tank to a microfluidic channel, the microfluidic channel forming part of a microfluidic magnetic separator.

10. The device of claim 9, wherein at least one of:
the breath capture portion further comprises a window configured to pass fluorescent light from the microfluidic channel to outside the breath capture portion; and
the window is also configured to allow light from outside the breath capture portion to illuminate the microfluidic channel.

11. The device of claim 10, further comprising a detection system configured to:
detect, through the window, fluorescence over a range of fluorescently labeled particle displacements in the microfluidic channel; and
provide, through the window, light to the microfluidic channel.

12. The device of claim 11, wherein the detection system comprises an LED detection system that illuminates the fluorescently labeled particles with LED light.

13. The device of claim 12, wherein:
the coronavirus is SARS-CoV-2; and
the first portion of SARS-CoV-2 to which the first coating binds and the second portion of SARS-CoV-2 to which the second coating binds are different epitopes of a SARS-CoV-2 spike protein.

14. The device of claim 13, wherein the second coating comprises angiotensin converting enzyme 2 (ACE2).

15. The device of claim 9, wherein at least one of:
at least one surface of the inlet tube and outlet tube is hydrophobic;
the device comprises a filter positioned to remove debris from the potentially pathogenic sample; and
the device comprises an exhaust filter that removes pathogen from vapor to be expelled from the device.

16. The device of claim 9, wherein at least a part of the breath capture portion is disposable.

17. The device of claim 16, wherein at least one of:
the disposable portion is the mouthpiece;
the mouthpiece is detachable from the breath capture portion; and
the entire breath capture portion is disposable.

18. The device of claim 9 further comprising:
a base, separate from the breath capture portion, comprising:
an interface for physically accommodating at least a portion of the breath capture portion;
electronics configured to obtain fluorescence data from the breath capture portion; and
a communications port for transferring communications based on the fluorescence data.

19. The device of claim 18, wherein at least one of:
the electronics comprise a detection system for detecting the fluorescence data;
the electronics comprise at least one of a video camera and a photosensor;
the at least one of a video camera and a photosensor is positioned to detect fluorescence over a range of fluorescently labeled particle displacements within the microfluidic channel;
the electronics is configured to run software to analyze images of the fluorescence;
the communications port comprises at least one of an ethernet port, a Bluetooth connection, a WiFi connection, a mobile phone connection, a bar-code reader or other method to correlate patient and breath sample, and an optical display on the base;
the base further comprises a piston positioned to be in mechanical communication with a part of the breath capture portion;
the part of the breath capture portion in mechanical communication with the piston comprises a plunger configured to displace the potentially pathogenic sample within the breath capture portion;
the piston is configured to actuate the plunger to displace the potentially pathogenic sample within the breath capture portion;
the base further comprises a motor configured to actuate the piston; and
the microfluidic magnetic separator has a High Gradient Magnetic Separation (HGMS) configuration.

20. The device of claim 19, wherein the microfluidic magnetic separator has an Open Gradient Magnetic Separation (OGMS) configuration.
